(19) United States
(12) Patent Application Publication
(10) Pub. No.: US 2022/0297157 A1
(43) Pub. Date: Sep. 22, 2022

(54) DNA SEQUENCING ASSEMBLIES AND MOLD ASSEMBLIES HAVING IMPROVED STABILITY IN AGGRESSIVE ENVIRONMENTS

(71) Applicant: Aculon, Inc., San Diego, CA (US)

(72) Inventor: Eric Hanson, Encinitas, CA (US)

(21) Appl. No.: 17/714,994

(22) Filed: Apr. 6, 2022

Related U.S. Application Data
(62) Division of application No. 15/662,377, filed on Jul. 28, 2017, now abandoned.
(60) Provisional application No. 62/367,783, filed on Jul. 28, 2016.

(51) Int. Cl. B05D 7/00; G16B 30/00; C03C 17/42

(52) U.S. Cl. CPC B05D 7/56; B05D 7/52; G16B 30/00; C03C 17/42; B05D 1/18

(57) ABSTRACT

Assemblies for DNA sequencing are provided comprising: (a) a substrate in the form of a flat plate; (b) a first coating layer applied to at least one surface of the substrate; and (c) a second coating layer applied to at least one surface of the first coating layer. The first coating layer comprises at least one of chromium oxide, tantalum pentoxide, Ta₂O₃ and TaO₂, and the second coating layer comprises a silane compound. Also provided are mold assemblies comprising: (a) a substrate having an interior surface and an exterior surface; and (b) a mold release component comprising: (i) a first coating layer similar to that above, applied to the interior surface of the substrate; and (ii) a second coating layer applied to the first coating layer; wherein the second coating layer comprises an organo-silicon compound comprising at least one of a trihalosilane, a tetrahalosilane, an organosilane, and polymers thereof.

### CROSS-REFERENCE TO RELATED APPLICATION

This application is a divisional of U.S. patent application Ser. No. 15/662,377, filed Jul. 28, 2017, and titled “COATED ARTICLES HAVING IMPROVED STABILITY IN AGGRESSIVE ENVIRONMENTS”, which claims priority from provisional U.S. Patent Application Ser. No. 62/367,783, filed Jul. 28, 2016, and titled “COATED ARTICLES HAVING IMPROVED STABILITY IN AGGRESSIVE ENVIRONMENTS”, both of which are incorporated herein by reference in their entireties.

### BACKGROUND OF THE INVENTION

### Field of the Invention

The present invention relates to assemblies for DNA sequencing demonstrating inhibited substrate degradation and mold assemblies demonstrating excellent mold release properties.

### Background of the Invention

Silicon-based oxides such as glass are generally susceptible to attack by water, especially at high or low pH. Because glass is used in a variety of industrial applications (many of which involve contact with water), this detriment can be especially problematic. In many cases, coatings are deposited onto glass surfaces (such as polyurethanes, epoxies, silanes, etc.) and suffer from the poor hydrolytic stability of the underlying oxide, resulting in delamination.

It would be desirable to provide articles having coatings that effectively act as a barrier to hydrolytic and other chemically aggressive attacks, such that subsequently applied coatings do not suffer from delamination problems.

### SUMMARY OF THE INVENTION

Assemblies for DNA sequencing are provided comprising:
(a) a substrate in the form of a flat plate having two opposing surfaces;
(b) a first coating layer applied to at least one surface of the substrate; wherein the first coating layer comprises at least one of chromium oxide, tantalum pentoxide, Ta₂O₃ and TaO₂; and
(c) a second coating layer applied to at least one surface of the first coating layer; wherein the second coating layer comprises a silane compound comprising at least one of a trihalosilane, a tetrahalosilane, an organosilane, and polymers thereof.

Also provided are mold assemblies comprising:
(a) a substrate having an interior surface and an exterior surface; and
(b) a mold release component comprising:
(i) a first coating layer applied to at least a portion of the interior surface of the substrate; wherein the first coating layer comprises at least one of chromium oxide, tantalum pentoxide, Ta₂O₃ and TaO₂; and
(ii) a second coating layer applied to at least a portion of the first coating layer; wherein the second coating layer comprises an organo-silicon compound comprising at least one of a trihalosilane, a tetrahalosilane, an organosilane, and polymers thereof.

### DETAILED DESCRIPTION OF THE INVENTION

Other than in any operating examples, or where otherwise indicated, all numbers expressing quantities of ingredients, reaction conditions and so forth used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used in this specification and the appended claims, the articles “a,” “an,” and “the” include plural referents unless expressly and unequivocally limited to one referent.

The various aspects and examples of the present invention as presented herein are each understood to be non-limiting with respect to the scope of the invention.

As used in the following description and claims, the following terms have the meanings indicated below:

The term “reactive” refers to a functional group capable of undergoing a chemical reaction with itself and/or other functional groups spontaneously or upon the application of heat or in the presence of a catalyst or by any other means known to those skilled in the art.

The terms “on”, “appended to”, “affixed to”, “bonded to”, “adhered to”, or terms of like import means that the designated item, e.g., a coating, film or layer, is either directly connected to the object surface, or indirectly connected to the object surface, e.g., through one or more other coatings, films or layers.

The coated articles of the present invention comprise a substrate (a). Substrates suitable for use in the preparation of the coated articles of the present invention can include metals such as aluminum, copper, or stainless steel; plastic substrates; or non-plastic substrates such as glass. Glass substrates may comprise any type of glass such as at least one of fused quartz glass, soda lime silica glass, sodium borosilicate glass, lead oxide glass, and aluminosilicate glass.

Suitable examples of plastic substrates include organic polymers such as polyol(allyl carbonate) monomers, e.g., allyl diglycol carbonates such as diethylene glycol bis(allyl carbonate); polyurea-polyurethane (polyurea urethane) polymers, which are prepared, for example, by the reaction of a polyurethane prepolymer and a diamine curing agent; polyol(meth)acryloyl terminated carbonate monomer; diethylene glycol dimethacrylate monomers; ethoxylated phenol methacrylate monomers; diisopropenyl benzene monomers; ethoxylated trimethylol propane triacrylate monomers; ethylene glycol bismethacrylate monomers; poly(ethylene glycol) bismethacrylate monomers; urethane acrylate monomers; poly(ethoxylated Bisphenol A dimethacrylate); poly(vinyl acetate); poly(vinyl alcohol); poly(vinyl chloride); poly(vinylidene chloride); polyethylene; polypropylene; polyurethanes; polythiourethanes; thermoplastic polycarbonates, such as the carbonate-linked resin derived from Bisphenol A and phosgene, one such material being sold under the trademark LEXAN; polyesters, such as the material sold under the trademark MYLAR; poly(ethylene terephthalate); polyvinyl butyral; poly(methyl methacrylate), such as the material sold under the trademark PLEXIGLAS, and polymers prepared by reacting polyfunctional isocyanates with polythiols or polyepisulfide monomers, either homopolymerized or co-and/or terpolymerized with polythiols, polyisocyanates, polyisothiocyanates and optionally ethylenically unsaturated monomers or halogenated aromatic-containing vinyl monomers. Also suitable are copolymers of such monomers and blends of the described polymers and copolymers with other polymers, e.g., to form interpenetrating network products.

The substrate may take any shape as desired for the intended application, such as flat, curved, bowl-shaped, tubular, or freeform. For example, the substrate may be in the form of a flat plate having two opposing surfaces, such as would be suitable for use in an assembly for DNA sequencing. When the substrate is intended for use as a mold, it may have any desired shape or configuration. Mold substrates usually have an “interior” or “molding” surface (i.e., the surface to be used to shape an article) and an exterior surface.

Prior to application of any coatings, the substrate may be cleaned such as by argon plasma treatment.

The coated articles of the present invention further comprise a first coating layer (b) applied to at least one surface of the substrate. The first coating layer comprises a material that inhibits degradation of the substrate. By “degradation” is meant loss of substrate material from the substrate surface such as by corrosion, dissolution, hydrolysis, or other chemical reactions in which the substrate material may be consumed. The first coating layer often comprises a metal oxide, such as at least one of chromium oxide, tantalum pentoxide, Ta₂O₃ and TaO₂. It has been discovered that these metal oxides effectively act as a barrier to hydrolytic attack of glass substrates. Often the first coating layer consists essentially of a metal oxide and is essentially free of other film-forming compounds such as silanes or organic polymers. As used throughout this specification, including the claims, by “essentially free” is meant that if a compound is present in the composition, it is present incidentally in an amount less than 0.1 percent by weight, usually less than trace amounts.

The first coating layer may be applied to the substrate by vapor deposition, atomic layer 3-phase deposition, thermal evaporation, or as a sol gel. A sol-gel may be applied to the substrate by one or more of a number of methods such as spraying, dipping (immersion), spin coating, or flow coating onto a surface thereof.

Sol-gels are dynamic systems wherein a solution (“sol”) gradually evolves into a gel-like two-phase system containing both a liquid phase and solid phase, whose morphologies range from discrete particles to continuous polymer networks within the continuous liquid phase.

The first coating layer typically has a dry film thickness (DFT) of less than 1000 nm, such as less than 500 nm. A DFT of 400 to 600 Angstroms is typical for vapor deposited layers.

The coated articles of the present invention further comprise a second coating layer (c) that is different from the first coating layer and is applied to at least one surface of the first coating layer. The second coating layer comprises an organo-silicon compound. Suitable organo-silicon compounds include trihalosilanes, tetrahalosilanes such as perfluorosilane, organosilanes, and polymers (including sol-gels) thereof. Mixtures of compounds may also be used. Often the second coating layer is essentially free of metal oxides.

Suitable trihalosilanes include alkyltrihalosilanes, such as alkyltrifluorosilanes, alkyltrichlorosilanes, and alkyltribromosilanes. Examples of suitable alkyltrichlorosilanes include methyltrichlorosilane, vinyltrichlorosilane, ethyltrichlorosilane, n-propyltrichlorosilane, i-propyltrichlorosilane, γ-chloropropyltrichlorosilane, i-butyltrichlorosilane, n-butyltrichlorosilane, pentyltrichlorosilane, hexyltrichlorosilane, heptyltrichlorosilane, n-octyltrichlorosilane, i-octyltrichlorosilane, hexadecyltrichlorosilane, 10-undecenyltrichlorosilane, 13-tetradecenyltrichlorosilane, 14-pentadecenyltrichlorosilane, 15-hexadecenyltrichlorosilane, n-octadecyltrichlorosilane and n-hexadecyltrichlorosilane.

Suitable organosilanes typically have the structure:

SiR₄

wherein each R independently comprises H or an organic group selected from linear, branched, or cyclic alkyl having 1 to 12 carbon atoms; alkoxy; and polyalkoxy; and wherein at least one R comprises an organic group. Alkyl groups may be substituted with functional groups such as halo-, aldehyde, epoxy, hydroxyl, and the like, for particular applications. Examples of suitable organosilanes include trimethoxysilane and glycidylpropyl trimethoxysilane. An example of a polymeric organosilane is trimethoxysilyl-terminated polyperfluorosilane.

The organo-silicon compound may be dissolved in a solvent such as an aprotic solvent. An exemplary solvent is 3-ethoxyperfluoro(2-methylhexane) (HFE 7500, available from 3M). The second coating layer may be applied to the first coating layer by one or more of a number of methods such as spraying, dipping (immersion), spin coating, or flow coating onto a surface thereof. Immersion is used most often. The second coating layer may also be applied as a sol-gel layer, deposited onto the first coating layer from, for example, a solution of hydrolyzed trialkoxysilane in an alcohol having 1 to 6 carbon atoms, such as isopropanol.

After application of the second coating layer, the coated article may be subjected to elevated temperatures, such as at least 80° C., or at least 120° C., for a time sufficient to at least partially cure the second coating layer. Durations of at least 30 minutes, depending on the temperature, such as at least 2 hours, are typical.

The term “cure”, “cured” or similar terms, as used in connection with a cured or curable composition, e.g., a “cured composition” of some specific description, means that at least a portion of any polymerizable and/or crosslinkable components that form the curable composition is polymerized and/or crosslinked. Additionally, curing of a composition refers to subjecting said composition to curing conditions such as those listed above, leading to the reaction of the reactive functional groups of the composition. The term “at least partially cured” means subjecting the composition to curing conditions, wherein reaction of at least a portion of the reactive groups of the composition occurs. The composition can also be subjected to curing conditions such that a substantially complete cure is attained and wherein further curing results in no significant further improvement in physical properties, such as hardness.

The second coating layer typically has a final dry film thickness (DFT) of 4-10 nm.

The coated articles of the present invention may further comprise (d) a third coating layer applied to at least one surface of the second coating layer. The third coating layer may comprise any film-forming resin known in the art of surface coatings; the third coating layer often comprises a polyurethane or polyepoxide, depending on the application.

The coated articles of the present invention often demonstrate a water contact angle (WCA) of at least 110′. The water contact angle can be determined using a contact angle goniometer such as a TANTEC contact angle meter Model CAM-MICRO. After immersion of the coated article in an aqueous solution of NaOH at pH 12 for 24 hours at 60° C., the coated article continues to demonstrate a WCA of at least 110°. Note that this result is typically observable even after up to 60 days immersion.

The coated articles of the present invention are suitable for use as molds, demonstrating excellent mold release properties. The present invention is thus further drawn to a mold assembly comprising:
(a) a substrate having an interior surface and an exterior surface; and
(b) a mold release component comprising:
(i) a first coating layer applied to at least a portion of the interior surface of the substrate; wherein the first coating layer comprises a metal oxide; and
(ii) a second coating layer applied to at least a portion of the first coating layer; wherein the second coating layer comprises a silane. having reactive terminal groups.

Suitable substrates include any of those disclosed above. Likewise, suitable metal oxides for use in the first coating layer (i) may be any of those disclosed above.

The second coating layer (ii) comprises a silane, such as any of those disclosed above, optionally having terminal groups such as fluorosilane or having reactive terminal groups. In particular examples, the terminal groups on the silane can initiate a living polymerization process. A living polymerization process is a chain-growth polymerization that propagates with essentially no chain transfer and essentially no chain termination. An example of a living polymerization process is atom transfer radical polymerization (ATRP). A group that can initiate a living polymerization process is a radically transferable atom or group, typically a halo group such as a bromo-group.

Mold assemblies of the present invention may be used for contact lenses, wherein the interior surface of the substrate is concave.

The coated articles of the present invention may also be used as substrates in an assembly for DNA sequencing. The present invention is further drawn to assembly for DNA sequencing comprising:
(a) a substrate;
(b) a first coating layer applied to at least one surface of the substrate; wherein the first coating layer comprises a metal oxide; and
(c) a second coating layer applied to at least one surface of the first coating layer; wherein the second coating layer comprises an organo-silicon compound.

Suitable substrates include any of those disclosed above. Like wise, suitable metal oxides for use in the first coating layer (a), and suitable organo-silicon compounds for use in the second coating layer (b) may be any of those disclosed above. Often the organo-silicon compound in the second coating layer comprises a silane having terminal functional groups that may either allow for the adhesion of biological materials, selected from azide, alkyne, isocyanate, epoxy, aldehyde, carboxylic acid, amine, phosphate, and hydroxyl groups, allowing for “anchoring” of DNA on the coated substrate surface for sequencing purposes. Alternatively, terminal functional groups may be selected to prevent adhesion of biological materials.

The following examples are intended to illustrate various embodiments of the invention, and should not be construed as limiting the invention in any way.

### EXAMPLES

Example 1A is a comparative example using an untreated glass substrate. Example 1B according to the present invention uses a glass substrate coated with 500 Angstroms of tantalum oxide (Ta₂O₅) as a first coating layer, applied via physical vapor deposition by UHV Sputtering (Morgan Hill, Calif.). Test samples were first cleaned with 15 min Ar+plasma (700 mTorr, power setting on “High”) then coated with a hydro-oleophobic silane layer by immersion in 0.1% trimethoxysilyl-terminated polyperfluorosilane in HFE-7500 fluorosolvent (available from 3M), followed by curing at 120° C. for 2 hours. Excess silane was then removed by rinsing with HFE-7500 solvent then water/oil contact angles were measured:

### Example 1A (Comparative): WCA=112, OCA=70

### Example 1B: WCA=115, OCA=74

These samples were then placed in an aqueous, pH 12 solution (NaOH) at 60° C. for one hour, removed and rinsed with water followed by WCA/OCA measurements.

### Example 1A (Comparative): WCA=<10 (Droplet Spread Immediately), OCA=10-20 Range

### Example 1B: WCA=114, OCA=74

The results show that the substrates coated in accordance with the present invention demonstrate improved hydrolytic stability of glass in caustic environments.

Example 2A is a comparative example using an untreated glass substrate. Example 2B according to the present invention uses a glass substrate coated with 500 Angstroms of tantalum oxide (Ta₂O₅) as a first coating layer, applied via physical vapor deposition by UHV Sputtering (Morgan Hill, Calif.). The glass substrates of Examples 2A and 2B were cleaned as in Example 1 followed by application of 0.1% glycidylpropyltrimethoxysilane in toluene, drying at room temperature (ca. 25° C.) drying for 15 minutes, then curing at 80° C. for 30 minutes, followed by rinsing with fresh toluene. Several of these samples were then exposed to 200 nM DNA (˜10 kDa, —NH₂ labelled) in distilled water for 16 hours at room temperature followed by rinsing with deionized water, immersion in deionized water for 3 hours, then rinsing with deionized water and drying. Half of these samples underwent an additional treatment (to see if the linkages would remain present under conditions that should hydrolyze the glass surface) by immersion in 2 μM DNA (˜10 kDa, —NH₂ labelled) in deionized water at 60° C. followed by deionized water rinse, immersion in deionized water for 3 hours, then deionized water rinse. XPS (X-ray photoelectron spectroscopy) was used to analyze the samples specifically for the presence of phosphorus, which is abundant in DNA.

| | Percent by weight Phosphorus |
|---|---|
| Example 2A (Comparative) | |
| After plasma cleaning | <0.1 |
| After application of | <0.1 |
| glycidylpropyltrimethoxysilane | |
| After exposure to 200 nM DNA | 0.1 |
| After exposure to 2 μM DNA at 60° C. | Not detected |
| Example 2B | |
| After plasma cleaning | <0.1 |
| Aftwer application of | <0.1 |
| glycidylpropyltrimethoxysilane | |
| After exposure to 200 nM DNA | 0.5 |
| After exposure to 2 μM DNA at 60° C. | 0.2 |

The results show that the substrates coated in accordance with the present invention demonstrate improved hydrolytic stability of glass, allowing for adhesion of DNA molecules even under conditions that normally cause hydrolysis of glass substrates.

Whereas particular embodiments of this invention have been described above for purposes of illustration, it will be evident to those skilled in the art that numerous variations of the details of the present invention may be made without departing from the scope of the invention as defined in the appended claims.

What is claimed is:

1. An assembly for DNA sequencing comprising:
(a) a substrate in the form of a flat plate having two opposing surfaces;
(b) a first coating layer applied to at least one surface of the substrate; wherein the first coating layer comprises at least one of chromium oxide, tantalum pentoxide, Ta₂O₃ and TaO₂; and
(c) a second coating layer applied to at least one surface of the first coating layer; wherein the second coating layer comprises a silane compound comprising at least one of a trihalosilane, a tetrahalosilane, an organosilane, and polymers thereof.

2. The assembly of claim 1 wherein the substrate comprises at least one of fused quartz glass, soda lime silica glass, sodium borosilicate glass, lead oxide glass, aluminosilicate glass, an organic polymer, and a metal.

3. The assembly of claim 1, wherein the first coating layer has a dry film thickness of less than 1000 nm.

4. The assembly of claim 1, wherein the silane compound in the second coating layer has terminal functional groups selected from azide, alkyne, isocyanate, epoxy, aldehyde, carboxylic acid, amine, phosphate, and hydroxyl groups.

5. The assembly of claim 4, wherein the silane compound in the second coating layer comprises glycidylpropyl trimethoxysilane.

6. The assembly of claim 1, wherein the silane compound in the second coating layer has terminal functional groups that prevent adhesion of biological materials to the substrate.

7. A mold assembly comprising:
(a) a substrate having an interior surface and an exterior surface; and
(b) a mold release component comprising:
(i) a first coating layer applied to at least a portion of the interior surface of the substrate; wherein the first coating layer comprises at least one of chromium oxide, tantalum pentoxide, Ta₂O₃ and TaO₂; and
(ii) a second coating layer applied to at least a portion of the first coating layer; wherein the second coating layer comprises an organo-silicon compound comprising at least one of a trihalosilane, a tetrahalosilane, an organosilane, and polymers thereof.

8. The assembly of claim 7 wherein the substrate comprises at least one of fused quartz glass, soda lime silica glass, sodium borosilicate glass, lead oxide glass, aluminosilicate glass, an organic polymer, and a metal.

9. The assembly of claim 7, wherein the first coating layer has a dry film thickness of less than 1000 nm.

10. The assembly of claim 7 wherein the organo-silicon compound in the second coating layer comprises at least one of trimethoxysilane, glycidylpropyl trimethoxysilane, and trimethoxysilyl-terminated polyperfluorosilane.

11. The assembly of claim 7 wherein the organo-silicon compound in the second coating layer comprises at least one of an alkyltrifluorosilane, an alkyltribromosilane, methyltrichlorosilane, vinyltrichlorosilane, ethyltrichlorosilane, n-propyltrichlorosilane, i-propyltrichlorosilane, γ-chloropropyltrichlorosilane, i-butyltrichlorosilane, n-butyltrichlorosilane, pentyltrichlorosilane, hexyltrichlorosilane, heptyltrichlorosilane, n-octyltrichlorosilane, i-octyltrichlorosilane, hexadecyltrichlorosilane, 10-undecenyltrichlorosilane, 13-tetradecenyltrichlorosilane, 14-pentadecenyltrichlorosilane, 15-hexadecenyltrichlorosilane, n-octadecyltrichlorosilane and n-hexadecyltrichlorosilane.

12. The assembly of claim 7 wherein the organo-silicon compound in the second coating layer has terminal functional groups that can initiate a living polymerization process.

13. The assembly of claim 7 wherein the interior surface of the substrate is concave.

14. The assembly of claim 13 wherein the assembly is a contact lens mold.

15. The assembly of claim 7, further comprising:
(d) a third coating layer applied to at least one surface of the second coating layer; wherein the third coating layer comprises a polyurethane.

16. The assembly of claim 7, wherein the assembly demonstrates a water contact angle (WCA) of at least 110°, determined using a contact angle goniometer.

17. The assembly of claim 16, wherein after immersion of the assembly in an aqueous solution of NaOH at pH 12 for 24 hours at 60° C., the assembly demonstrates a water contact angle (WCA) of at least 110°, determined using a contact angle goniometer.

18. A mold assembly or an assembly for DNA sequencing comprising one of:
A) (i) a substrate in the form of a flat plate having two opposing surfaces;
(ii) a first coating layer applied to at least one surface of the substrate; wherein the first coating layer comprises at least one of chromium oxide, tantalum pentoxide, Ta₂O₃ and TaO₂; and
(iii) a second coating layer applied to at least one surface of the first coating layer; wherein the second coating layer comprises a silane compound comprising at least one of a trihalosilane, a tetrahalosilane, an organosilane, and polymers thereof; or
B) (i) a substrate having an interior surface and an exterior surface; and
(ii) a mold release component comprising:
(iii) a first coating layer applied to at least a portion of the interior surface of the substrate; wherein the first coating layer comprises at least one of chromium oxide, tantalum pentoxide, Ta₂O₃ and TaO₂; and
(iv) a second coating layer applied to at least a portion of the first coating layer; wherein the second coating layer comprises an organo-silicon compound comprising at least one of a trihalosilane, a tetrahalosilane, an organosilane, and polymers thereof.

19. The mold assembly or an assembly for DNA sequencing of claim 18, wherein the substrate comprises at least one of fused quartz glass, soda lime silica glass, sodium borosilicate glass, lead oxide glass, aluminosilicate glass, an organic polymer, and a metal.

20. The mold assembly or an assembly for DNA sequencing of claim 18, wherein the first coating layer has a dry film thickness of less than 1000 nm.
